SEQUENCE LISTING (14 sequences)

SEQ ID NO: 1 - protein, 214 aa, Artificial sequence
  Y0101-VL
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial sequence
  Y0101-VH
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial sequence
  TKKK-VL
DIQLTQSPSSLSASVGDRVTITCSATKKIKNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 214 aa, Artificial sequence
  TKKT-VL
DIQLTQSPSSLSASVGDRVTITCSATKKITNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 231 aa, Artificial sequence
  T28D/S100aR-VH
EVQLVESGGGLVQPGGSLRLSCAASGYDFTNYGMNWVRQAPGKGLEWVGWINTYTGEPTYAADFKRRFTFSLDTSKSTAYLQMNSLRAEDTAVYYCAKYPHYYGRSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 6 - protein, 11 aa, Artificial sequence
  TKKK as CDR L1
SATKKIKNYLN

SEQ ID NO: 7 - protein, 11 aa, Artificial sequence
  TKKT as CDR L1
SATKKITNYLN

SEQ ID NO: 8 - protein, 234 aa, Artificial sequence
  H97Y+VNERK-VH
EVQLVESGGGLVQPGGSLRLSCAASGYDFTNYGMNWVRQAPGKGLEWVGWINTYTGEPTYAADFKRRFTFSLDTSKSTAYLQMNSLRAEDTAVYYCAKYPYYYVNERKSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 9 - protein, 231 aa, Artificial sequence
  H97Y-VH
EVQLVESGGGLVQPGGSLRLSCAASGYDFTNYGMNWVRQAPGKGLEWVGWINTYTGEPTYAADFKRRFTFSLDTSKSTAYLQMNSLRAEDTAVYYCAKYPYYYGRSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 10 - protein, 234 aa, Artificial sequence
  VNERK-VH
EVQLVESGGGLVQPGGSLRLSCAASGYDFTNYGMNWVRQAPGKGLEWVGWINTYTGEPTYAADFKRRFTFSLDTSKSTAYLQMNSLRAEDTAVYYCAKYPHYYVNERKSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 11 - protein, 214 aa, Artificial sequence
  D3H44-VL
DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 225 aa, Artificial sequence
  D3H44-VH
EVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 13 - protein, 214 aa, Artificial sequence
  4D5-VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 228 aa, Artificial sequence
  4D5-VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

(19) United States
(12) Patent Application Publication
(10) Pub. No.: US 2008/0299115 A1
(43) Pub. Date: Dec. 4, 2008

(54) Antibody Variants with Faster Antigen Association Rates

(76) Inventors: Henry B. Lowman, El Granada, CA (US); Jonathan S. Marvin, New York, NY (US)

Correspondence Address: GENENTECH, INC., 1 DNA WAY, SOUTH SAN FRANCISCO, CA, 94080, US

(21) Appl. No.: 12/061,551

(22) Filed: Apr. 2, 2008

Related U.S. Application Data
(63) Continuation of application No. 11/537,851, filed on Oct. 2, 2006, now abandoned.
Application No. 11/537,851 is a continuation of application No. 10/364,953, filed on Feb. 11, 2003, now abandoned.
(60) Provisional application No. 60/355,895, filed on Feb. 11, 2002.
(60) Provisional application No. 60/409,685, filed on Sep. 10, 2002.

(51) Int. Cl. A61K 39/395; C07K 16/18; C12N 15/11; C12N 5/06; A61P 43/00; G01N 33/536; C12N 15/00; C12P 21/04

(52) U.S. Cl. 424/133.1; 530/387.1; 530/387.3; 435/69.6; 536/23.53; 435/320.1; 435/325; 436/536

(57) ABSTRACT

Antibody variants with faster antigen association rates are disclosed. The antibody variants have one or more amino acid alteration(s) in or adjacent to at least one hypervariable region thereof which increase charge complementarity between the antibody variant and an antigen to which it binds.

This is a continuation application claiming priority to U.S. application Ser. No. 10/364,953, filed Feb. 11, 2003, which is a non-provisional application claiming priority to U.S. Provisional Application Nos. 60/355,895, filed Feb. 11, 2002 and 60/409,685, filed Sep. 10, 2002, the entire disclosures of which are hereby incorporated by reference.

### BACKGROUND OF THE INVENTION

1. Field of the Invention

The invention herein pertains to antibody variants with faster antigen association rates. The antibody variants have one or more alterations in or adjacent to at least one hypervariable region thereof, where the alteration(s) increase charge complementarity between the antibody variant and an antigen to which it binds.

2. Description of Related Art

Antibodies are proteins, which exhibit binding specificity to a specific antigen. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in three segments called Complementarity Determining Regions (CDRs) both in the light chain and the heavy chain variable domains. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al., Sequences of Proteins of Immunological Interest, 5th Ed. Public Health Service, National Institutes of Health, Bethesda, Md. (1991)).

The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and several of these may be further divided into subclasses (isotypes), e.g. IgG1, IgG2, IgG3, and IgG4; IgA1 and IgA2. The heavy chain constant regions that correspond to the different classes of immunoglobulins are called α, δ, ε, γ, and μ, respectively. Of the various human immunoglobulin classes, only human IgG1, IgG2, IgG3 and IgM are known to activate complement.

The use of antibodies for the treatment of human diseases is rapidly increasing. One such therapeutically relevant antibody has been constructed to target vascular endothelial growth factor (VEGF) (Chen et al. Journal of Molecular Biology 293(4): 865-81 (1999); Kim et al. Nature 362(6423): 841-4 (1993); Muller et al. Structure 5(10): 1325-38 (1997); WO 96/30046; WO 98/45331; and WO 00/29584). VEGF specifically initiates blood vessel proliferation through its stimulation of the transmembrane receptors, Flt-1 and KDR (Ferrara, N. Current Topics in Microbiology & Immunology 237: 1-30 (1999)). Antagonists of VEGF have been demonstrated to suppress diseases, including cancer, in which uncontrolled angiogenesis contributes to the diseased state (Kim et al. Nature 362(6423): 841-4 (1993)).

In vivo, affinity maturation of antibodies is driven by antigen selection of higher affinity antibody variants which are made primarily by somatic hypermutagenesis. A “repertoire shift” also often occurs in which the predominant germline genes of the secondary or tertiary response are seen to differ from those of the primary or secondary response.

Various research groups have attempted to mimic the affinity maturation process of the immune system, by introducing mutations into antibody genes in vitro and using affinity selection to isolate mutants with improved affinity. Such mutant antibodies can be displayed on the surface of filamentous bacteriophage and antibodies can be selected by their affinity for antigen or by their kinetics of dissociation (off-rate) from antigen. Hawkins et al. J. Mol. Biol. 226:889-896 (1992). CDR walking mutagenesis has been employed to affinity mature human antibodies which bind the human envelope glycoprotein gp120 of human immunodeficiency virus type 1 (HIV-1) (Barbas III et al. PNAS (USA) 91: 3809-3813 (1994); and Yang et al. J. Mol. Biol. 254:392-403 (1995)); and an anti-c-erbB-2 single chain Fv fragment (Schier et al. J. Mol. Biol. 263:551567 (1996)). Antibody chain shuffling and CDR mutagenesis were used to affinity mature a high-affinity human antibody directed against the third hypervariable loop of HIV (Thompson et al. J. Mol. Biol. 256:77-88 (1996)). Balint and Larrick Gene 137:109-118 (1993) describe a technique they coin “parsimonious mutagenesis” which involves computer-assisted oligodeoxyribonucleotide-directed scanning mutagenesis whereby all three CDRs of a variable region gene are simultaneously and thoroughly searched for improved variants. Wu et al. affinity matured an αvβ3-specific humanized antibody using an initial limited mutagenesis strategy in which every position of all six CDRs was mutated followed by the expression and screening of a combinatorial library including the highest affinity mutants (Wu et al. PNAS (USA) 95: 6037-6-42 (1998)). Phage antibodies are reviewed in Chiswell and McCafferty TIBTECH 10:80-84 (1992); and Rader and Barbas III Current Opinion in Biotech. 8:503-508 (1997).

The affinity of a protein-ligand pair is described by the dissociation constant (Kd) and defined as the equilibrium distribution of unbound molecules to bound molecules in solution (Eq. 1). This relationship can also be defined by the ratio of the dissociation rate constant (off-rate constant, k₋₁) to the association rate constant (on-rate constant, k₁).

$$P + L {{\to }_{{←}_{{k}_{- 1}}}}^{ {k}_{1} } PL \\ {K}_{d} = \frac{[ P ] [ L ]}{[ PL ]} = \frac{{k}_{- 1}}{{k}_{1}} \qquad Eq . 1$$

Affinity differences among mutants of many protein-protein interactions (Voss, E. W. Journal of Molecular Recognition 6(2): 51-8 (1993)) are defined primarily by differences in their dissociation rates. This observation is consistent with mutations that increase affinity participating in direct contacts at the protein-protein interface, and dissociation rate constants being dependent on the breaking of favorable short range interactions. In contrast, association rate constants (k₁) are dependent on the frequency of collision between the two molecules (Z), and the efficiency with which each collision results in the formation of a complex. The latter in turn is dependent on a steric factor (p) to account for orientation requirement of the two molecules and the population of molecules with sufficient thermal activation energy (Fersht, A. R. (1985). Enzyme Structure and Mechanism, W. H. Freeman and Company, New York, N.Y.) (Eq. 2).

$${k}_{1} = Zp {e}^{\frac{- Ea}{RT}} \qquad Eq . 2$$

where Ea is the activation energy for formation of the complex, R is the universal gas constant, and T is the temperature (in Kelvins).

In theory, it is possible to increase the association rate through mutations that increase the rate of collision or efficiency of collision. It has been postulated that this can be achieved, without disrupting the short-range contacts that comprise the binding interface, by mutating residues at the periphery of the binding interface to generate favorable electrostatic steering forces (Berg & von Hippel (1996) Nat. Struct. Biol. 3:427-31; Radic et al. (1997) J. Biol. Chem. 272:23265-77; Selzer et al. (2000) Nat. Struct. Biol. 7:537-41. Investigations of this phenomenon have focused on Brownian dynamics simulations and complex computational analysis to solve the full non-linear Poisson-Boltzman equation for prediction of association rates in solutions of varying viscosity and salinity (Slagle et al. (1994) J. Biomolec. Struct. Dynam. 12:439-56; Kozack et al. (1995) Biophys. J. 68-807-14; Fogolari et al. (2000) Eur J. Biochem. 267:4861-9; Gabdoulline & Wade (2001) J. Mol. Biol. 306:1139-55). However, it has recently been shown that association rates can be predicted by calculating the electrostatic energy of interaction with a homogenous dielectric constant of 80 for the barnase-barstar complex (Schreiber & Fersht (1996) Nat. Struct. Biol. 3:427-31; Vijayakumar et al. (1998) J. Mol. Biol. 278:1015-24), TEM-lactamase-BLIP inhibitor complex (Selzer et al. (2000) Nat. Struct. Biol. 7:537-41), acethylcholinesterase-fasciculin complex (Radic et al. (1997) J. Biol. Chem. 272:23265-77, and the hirudin-thrombin complex (Jackman et al. (1992) J. Biol. Chem. 267:15375-83; Betz et al. (1991) Biochem. J. 275:801-3).

### SUMMARY OF THE INVENTION

The present invention provides a method of making an antibody variant of a parent antibody comprising a) identifying a target amino acid residue within the variable domain of the parent antibody, said target residue being 1) an exposed residue in solution; 2) in or adjacent to a hypervariable region; and 3) within about 20 Å of the antigen when the parent antibody is bound thereto; and b) substituting the target residue of step a) with a different replacement amino acid residue such that the charge complementarity between the antibody and antigen is increased. In one aspect, the method of the invention results in an antibody variant having a faster association rate with the antigen than the parent antibody. The invention further provides an antibody variant made according to the method of the preceding paragraph.

In addition, the invention provides an antibody variant which comprises an amino acid alteration in or adjacent to a hypervariable region thereof which increases charge complementarity between the antibody variant and an antigen to which it binds.

Various forms of the antibody variant are contemplated herein. For example, the antibody variant may be a full length antibody (e.g. having a human immunoglobulin constant region) or an antibody fragment (e.g. a Fab or F(ab′) 2). Furthermore, the antibody variant may be labeled with a detectable label, immobilized on a solid phase and/or conjugated with a heterologous compound (such as a cytotoxic agent).

Diagnostic and therapeutic uses for the antibody variant are contemplated. In one diagnostic application, the invention provides a method for determining the presence of an antigen of interest comprising exposing a sample suspected of containing the antigen to the antibody variant and determining binding of the antibody variant to the sample. For this use, the invention provides a kit comprising the antibody variant and instructions for using the antibody variant to detect the antigen.

The invention further provides: isolated nucleic acid encoding the antibody variant; a vector comprising the nucleic acid, optionally, operably linked to control sequences recognized by a host cell transformed with the vector; a host cell transformed with the nucleic acid; a process for producing the antibody variant comprising culturing this host cell so that the nucleic acid is expressed and, optionally, recovering the antibody variant from the host cell culture (e.g. from the host cell culture medium). The recovered antibody variant may be conjugated with a heterologous molecule, such as a cytotoxic agent or label.

The invention also provides a composition comprising the antibody variant and a pharmaceutically acceptable carrier or diluent. This composition for therapeutic use is sterile and may be lyophilized.

The invention further provides a method for treating a mammal comprising administering an effective amount of the antibody variant to the mammal.

The invention further provides a method for determining antigen association rate of an antibody comprising:

(1) combining antibody and antigen in solution, and then;

(2) determining formation of antibody-antigen complex over time.

### BRIEF DESCRIPTION OF THE DRAWINGS

FIGS. 1A-B depict alignments of light and heavy chain amino acid sequences for the parent antibody Y0101 Fab (SEQ ID NOs: 1 and 2, respectively); the altered light chain “S26T-Q27K-D28K-S30K” sequence (SEQ ID NO: 3); the altered light chain “S26T-Q27K-D28K-S30T” sequence (SEQ ID NO: 4); and altered heavy chain “T28D-S100aR” sequence (SEQ ID NO: 5). In FIGS. 1A-B the numbering is sequential, rather than according to the Kabat numbering system. Hence, for the heavy chain mutant, the S100aR mutation (Kabat numbering system) is mutation S105R (sequential numbering system).

FIG. 2 represents fluorescence spectra. The emission spectra of ˜10 nM Fab Y0101 (dashed black), ˜120 nM VEGF (solid grey), and a mixture of 10 nM Fab with 120 nM VEGF (solid black). The sum of the individual spectra of the Fab and VEGF is shown in dashed grey.

FIG. 3 represents raw kinetic data. The rate of formation of the complex (AFluorescence) can be measured as a function of time with varying concentrations of VEGF (increasing in concentration from grey to black) and fit to a single exponential to determine the observed rate (k_{obs}).

FIG. 4 concerns calculation of k₁. Plotting the observed rate of formation of the complex (k_{obs}) against the concentration of VEGF used, permits pseudo-first order analysis to determine k₁, given by the slope of the plot. The data shown here is for the heavy chain mutant T28E.

FIG. 5 reveals a comparison of k_{obs} and k_{calc} for Fab Y0101 variants.

FIGS. 6A and 6B provide an alignment of the light chain and heavy chain sequences of the anti-VEGF variants “34-TKKT+H97Y+VNERK” (SEQ ID NOs:4 and 8, respectively); “34-TKKT+H97Y” (SEQ ID NOs:4 and 9, respectively); and “34-TKKT+VNERK” (SEQ ID NOs:4 and 10, respectively). Sequences of the parent antibody Y0101 is provided for comparison. Residues in bold and underlined indicate substitutions.

FIG. 7 illustrates the dependence of association rate on ionic strength. The association rate for Y0101 (filled circles) and the fast binding variant, “34-TKKT” ((V_{H}-(T28D,S100aR)+V_{L}-(S26T, Q27K, D28K, S30T)) (open squares) was measured as a function of salt concentration. The slopes (−U/RT) are −1.4 and 6.5, respectively, corresponding to U of +0.86 kcal mol for Y0101 and −4.0 kcal mol 1 for the fastest binding variant.

FIG. 8 provides amino acid sequences for the light and heavy chain variable domains of the humanized anti-TF antibody D3H44. Residues identified as potential On-RAMPS are indicated in bold and underlined.

FIG. 9 provides amino acid sequences for the light and heavy chain variable domains of the humanized anti-HER2 antibody 4D5. Residues identified as potential On-RAMPS are indicated in bold and underlined.

### DETAILED DESCRIPTION OF THE PREFERRED EMBODIMENTS

### I. Definitions

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “hypervariable region” when used herein refers to the regions of an antibody variable domain which are hypervariable in sequence and/or form structurally defined loops. The hypervariable region comprises amino acid residues from a “complementarity determining region” or “CDR” (i.e. residues 24-34 (“CDR L1”), 50-56 (“CDR L2”) and 89-97 (“CDR L3”) in the light chain variable domain and 31-35 (“CDR H1”), 50-65 (“CDR H2”) and 95-102 (“CDR H3”) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (“loop L1”), 50-52 (“loop L2”) and 91-96 (“loop L3”) in the light chain variable domain and 26-32 (“loop H1”), 53-55 (“loop H2”) and 96-101 (“loop H3”) in the heavy chain variable domain; Chothia and Lesk J. Mol. Biol. 196:901-917 (1987)). In both cases, the variable domain residues are numbered according to Kabat et al., supra. “Framework” or “FR” residues are those variable domain residues other than the hypervariable region residues as herein defined.

The expression “variable domain residue numbering as in Kabat” refers to the numbering system used for heavy chain variable domains or light chain variable domains from the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout this application refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

A “parent antibody” is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. Preferably the parent antibody is a chimeric, humanized or human antibody.

As used herein, “antibody variant” refers to an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. Identity or similarity with respect to this sequence is defined herein as the percentage of amino acid residues in the candidate sequence that are identical (i.e same residue) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity. The antibody variant is generally one which comprises one or more amino acid alterations in or adjacent to one or more hypervariable regions thereof.

An “amino acid alteration” refers to a change in the amino acid sequence of a predetermined amino acid sequence. Exemplary alterations include insertions, substitutions and deletions.

An “amino acid substitution” refers to the replacement of an existing amino acid residue in a predetermined amino acid sequence; with another different amino acid residue.

A “replacement” amino acid residue refers to an amino acid residue that replaces or substitutes another amino acid residue in an amino acid sequence. The replacement residue may be a naturally occurring or non-naturally occurring amino acid residue.

An “amino acid insertion” refers to the introduction of one or more amino acid residues into a predetermined amino acid sequence.

The amino acid insertion may comprise a “peptide insertion” in which case a peptide comprising two or more amino acid residues joined by peptide bond(s) is introduced into the predetermined amino acid sequence. Where the amino acid insertion involves insertion of a peptide, the inserted peptide may be generated by random mutagenesis such that it has an amino acid sequence which does not exist in nature.

An amino acid alteration “adjacent a hypervariable region” refers to the introduction or substitution of one or more amino acid residues at the N-terminal and/or C-terminal end of a hypervariable region, such that at least one of the inserted or replacement amino acid residue(s) form a peptide bond with the N-terminal or C-terminal amino acid residue of the hypervariable region in question.

A “naturally occurring amino acid residue,” is one encoded by the genetic code, generally selected from the group consisting of: alanine (Ala); arginine (Arg); asparagine (Asn); aspartic acid (Asp); cysteine (Cys); glutamine (Gln); glutamic acid (Glu); glycine (Gly); histidine (H is); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

A “non-naturally occurring amino acid residue” herein is an amino acid residue other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “exposed” amino acid residue is one in which at least part of its surface is exposed, to some extent, to solvent when present in a polypeptide (e.g. an antibody or polypeptide antigen) in solution. Preferably, the exposed amino acid residue is one in which at least about one third of its side chain surface area is exposed to solvent. Various methods are available for determining whether a residue is exposed or not, including an analysis of a molecular model or structure of the polypeptide.

A “charged” amino acid residue is one bearing a net overall positive charge or a net overall negative charge. Positively charged amino acid residues include arginine, lysine and histidine. Negatively charged amino acid residues include aspartic acid and glutamic acid.

The term “target antigen” herein refers to a predetermined antigen to which both a parent antibody and antibody variant as herein defined bind. The target antigen may be polypeptide, carbohydrate, nucleic acid, lipid, hapten or other naturally occurring or synthetic compound. Preferably, the target antigen is a polypeptide. While the antibody variant generally binds the target antigen with better binding affinity than the parent antibody, the parent antibody usually has a binding affinity (K_{d}) value for the target antigen of no more than about 1×10⁵M, and preferably no more than about 1×10⁶M.

By “association rate” herein is meant the on-rate constant (k₁) with which an antibody forms a complex with antigen in solution.

Herein, “dissociation rate” refers to the off-rate constant (k₋₁), or breaking of short range interactions between antibody and antigen.

By “charge complementarity” herein is meant the electrostatic interaction between amino acid residue(s) of the antibody and amino acid residue(s) of the antigen. The charge here refers to the local charge of the antigen in the vicinity of the amino acid residue(s) of the antibody when the antibody is bound to antigen. To increase charge complementarity of, for example, a positively charged antibody to a negatively charged antigen, certain negatively charged amino acid residue(s) in the antibody (e.g., D or E) is/are replaced which either neutral residue(s) (e.g., N or T) or positively charged residues (R or K) in order to neutralize or reverse the negative charge to better complement the negatively charged antigen.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Treatment” refers to both therapeutic treatment and prophylactic or preventative measures. Those in need of treatment include those already with the disorder as well as those in which the disorder is to be prevented.

A “disorder” is any condition that would benefit from treatment with the antibody variant. This includes chronic and acute disorders or diseases including those pathological conditions which predispose the mammal to the disorder in question.

“Mammal” for purposes of treatment refers to any animal classified as a mammal, including humans, domestic and farm animals, nonhuman primates, and zoo, sports, or pet animals, such as dogs, horses, cats, cows, etc.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

### II. Modes for Carrying out the Invention

The invention herein relates, at least in part, to a method for making an antibody variant. The parent antibody or starting antibody may be prepared using techniques available in the art for generating such antibodies. Exemplary methods for generating antibodies are described in more detail in the following sections. Moreover, the present application does not require actual physical production of the parent antibody, since one can use available information (e.g. amino acid sequence data) for an antibody of interest to generate the antibody variants herein.

The parent antibody is directed against a target antigen of interest. Preferably, the target antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated.

Where the antigen is a polypeptide, it may be a transmembrane molecule (e.g. receptor) or ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor; platelet-derived growth factor (PDGF); fibroblast growth factor such as aFGF and bFGF; epidermal growth factor (EGF); transforming growth factor (TGF) such as TGF-alpha and TGF-beta; insulin-like growth factor-I and -II (IGF-I and IGF-II); des(1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Preferred molecular targets for antibodies encompassed by the present invention include CD proteins such as CD3, CD4, CD8, CD19, CD20 and CD34; members of the ErbB receptor family such as the EGF receptor, HER2, HER3 or HER4 receptor; cell adhesion molecules such as LFA-1, Mac1, p150,95, VLA-4, ICAM-1, VCAM and αv/β3 integrin including either alpha or beta subunits thereof (e.g. anti-CD11a, anti-CD18 or anti-CD11b antibodies); growth factors such as VEGF and TF; IgE; blood group antigens; flk2/flt3 receptor; obesity (OB) receptor; mp1 receptor; CTLA-4; protein C etc.

The antigen used to generate an antibody may be isolated from a natural source thereof, or may be produced recombinantly or made using other synthetic methods. Alternatively, cells comprising native or recombinant antigen can be used as immunogens for making antibodies.

The parent antibody may have pre-existing strong binding affinity for the target antigen. For example, the parent antibody may bind the antigen of interest with a binding affinity (K_{d}) value of no more than about 1×10⁻⁷ M, preferably no more than about 1×10⁻⁸ M and most preferably no more than about 1×10⁻⁹ M.

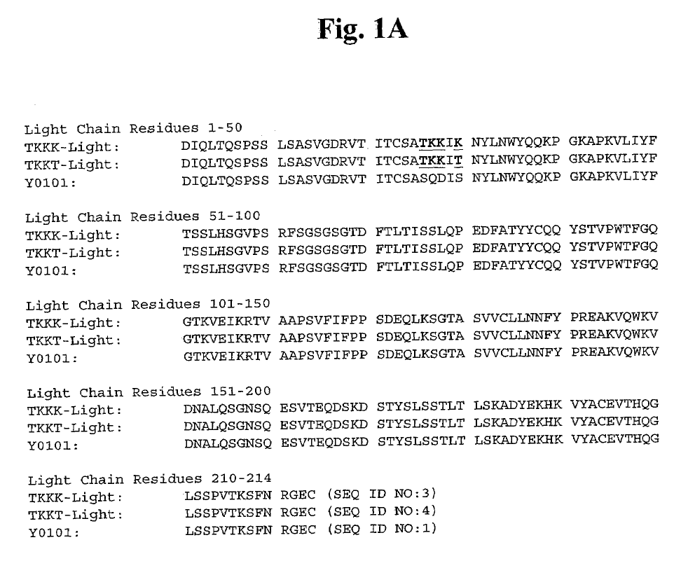

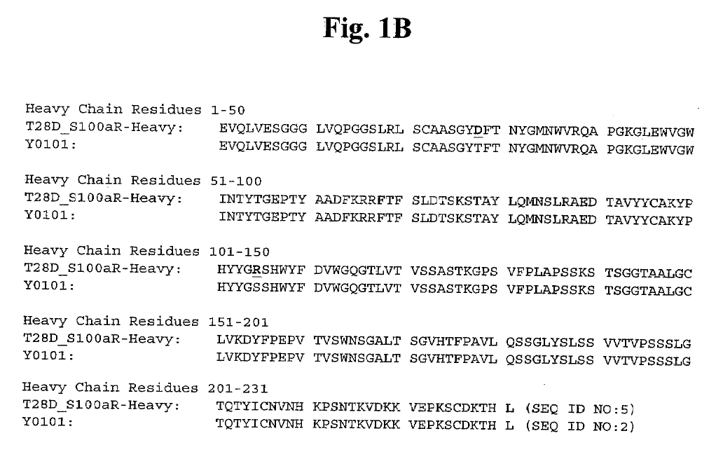

The parent antibody is preferably a chimeric (e.g. humanized) or human antibody. The chimeric, humanized or human antibody is optionally also an “affinity matured” antibody. Techniques for affinity maturing an antibody are referred to in the section under the heading “Description of Related Art” herein. In one embodiment, the parent antibody is an antibody fragment, or an antibody fragment (e.g. a Fab fragment) of a whole antibody is prepared for ease of screening recombinantly produced variants. Preferably, the parent antibody and antibody variant bind vascular endothelial growth factor (VEGF). An exemplary parent antibody comprises the light and heavy chain variable domains of an anti-VEGF antibody such as Y0101 (FIGS. 1A-B herein); Y0317 (WO98/45331, expressly incorporated herein by reference); humanized anti-VEGF F(ab)-12 (WO98/45331, expressly incorporated herein by reference); Y0192 (WO98/45331, expressly incorporated herein by reference); Y0238-3 (WO98/45331, expressly incorporated herein by reference); Y0239-19 (WO00/29584, expressly incorporated herein by reference); Y0313-2 (WO00/29584, expressly incorporated herein by reference) or VNERK mutant (WO00/29584, expressly incorporated herein by reference).

The antibody variant herein preferably displays a faster antigen association rate compared to the parent antibody. The association rate can be determined by any method in which formation of the complex may be observed as a function of time. The most widely used method is BIAcore® analysis, in which one measures the association of the antibody to an antigen that has been immobilized on a biosensor surface (reviewed by Rich & Myszka, Curr. Opin. Biotechnol. 11:54-61 (2000)). Alternatively, the association rate is measured in solution (rather than on a solid surface) by mixing antigen and antibody and measuring the rate of formation of the complex as a function of the concentration of antigen as in the Example herein. In this case, various detection methods are possible, including measurements of fluoresecence by intrinsic or artificial fluorophores (reviewed by Linthicum et al., Comb. Chem. High Throughput Screen 4:439-449 (2001). Preferably the association rate is determined according to the methodology in the Example herein. Most preferably, the association rate of the antibody variant is from about 5 fold, or from about ten fold (e.g. up to about 1000 fold, or up to about 10,000 fold) faster than that of the parent antibody.

The antibody variant further generally has a stronger binding affinity for the target antigen than the parent antibody. Antibody “binding affinity” may be determined by equilibrium methods (e.g. enzyme-linked immunoabsorbent assay (ELISA) or radioimmunoassay (RIA)), or kinetics (e.g. BIACOR™ analysis), for example. The antibody variant preferably has a binding affinity for the target antigen which is at least about two fold stronger, preferably at least about five fold stronger, and preferably at least about ten fold or 100 fold stronger (e.g. up to about 1000 fold or up to about 10,000 fold stronger binding affinity), than the binding affinity of the parent antibody for the antigen. The enhancement in binding affinity desired or required may depend on the initial binding affinity of the parent antibody.

Also, the antibody may be subjected to other “biological activity assays”, e.g., in order to evaluate its “potency” or pharmacological activity and potential efficacy as a therapeutic agent. Such assays are known in the art and depend on the target antigen and intended use for the antibody. Examples include the keratinocyte monolayer adhesion assay and the mixed lymphocyte response (MLR) assay for CD11a (see WO98/23761); tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); agonistic activity or hematopoiesis assays (see WO 95/27062); tritiated thymidine incorporation assay; and alamar blue assay to measure metabolic activity of cells in response to a molecule such as VEGF. The antibody variant preferably has a potency in the biological activity assay of choice which is at least about two fold greater (e.g. from about two fold to about 1000 fold or even to about 10,000 fold improved potency), preferably at least about 20 fold greater, more preferably at least about 50 fold greater, and sometimes at least about 100 fold or 200 fold greater, than the biological activity of the parent antibody in that assay.

The present invention provides a systematic method of making antibody variants that can be screened for improved function (e.g. for improved association rate and/or affinity). Preferably, one will evaluate available information concerning the antibody-antigen to determine candidate amino acid alteration(s) in the antibody that increase charge complementarity between the antibody and antigen. The molecular model may be obtained from an X-ray crystal or nuclear magnetic resonance (NMR) structure of this complex. See, e.g., Amit et al. Science 233:747-753 (1986); and Muller et al. Structure 6(9): 1153-1167 (1998)). Alternatively, computer programs can be used to create molecular models of antibody/antigen complexes (see, e.g., Levy et al. Biochemistry 28:7168-7175 (1989); Bruccoleri et al. Nature 335: 564-568 (1998); and Chothia et al. Science 233: 755-758 (1986)), e.g., where a crystal structure is not available.

In one embodiment, the alteration involves insertion of one or more charged amino acid residues in or adjacent to one or more hypervariable regions of the parent antibody. In this embodiment, the inserted residue(s) usually do not bind antigen as determined by analyzing the antibody-antigen complex. Generally, from about one to about twenty, or up to about forty, amino acid residues which increase charge complementarity may be inserted.

In the most preferred embodiment, the alteration involves substitution of one or more target residues in or adjacent to one or more hypervariable regions. According to this embodiment of the invention, the target residues may be selected as follows:

1) Preferably the residue is exposed in solution, e.g. has at least one third of its side chain surface area exposed to solvent. Without being bound to any one theory, this is thought to avoid possibly destabilizing the antibody through mutation of buried residues.
2) Desirably, the residue is within at least about 20 Å (preferably within about 16 Å) of antigen in the bound state, as electrostatic attractive forces may decay as a function of distance.
3) Preferably, the residue is not in direct contact with the antigen in the bound state, as mutation of direct contact residues may possibly destabilize the bound complex.
4) Preference is given to those residues that are within the hypervariable regions or complementarity determining regions (CDRs) over those that are not, as there are indications that such regions are less likely to induce an immunogenic response in patients.
5) Generally, only those residues for which it is possible to increase the charge complementarity between the antibody and the antigen are considered for alteration.

Hence, according to the preferred embodiment of the invention that is further illustrated in Example, one identifies one or more exposed hypervariable region amino acid residue(s) within about 20 Å of the antigen when the parent antibody is bound thereto, and substitutes one or more of those exposed residue(s) with a neutral or oppositely charged replacement amino acid residue.

While the present invention contemplates single amino acid substitutions according to the criteria herein, preferably two or more substitutions are combined, e.g. from about two to about ten or about twenty substitutions per variable domain (i.e. up to about twenty or about forty, respectively, amino acid substitutions for both variable domains). The alterations herein that increase charge complementarity between the antibody and antigen, may be combined with other amino acid sequence alterations in hypervariable regions or amino acid sequence alterations in other regions of the antibody.

In one embodiment, the hypervariable region with alteration(s) according to the invention herein is selected from the group consisting of CDR L1, CDR L2, loop H1 and CDR H3, and most preferably CDR L1. Moreover, alterations in two or more hypervariable regions, e.g. in two or more of CDR L1, CDR L2, loop H1 and CDR H3, may be combined. For instance, the antibody variant may comprise a light chain variable domain with one or more alterations in CDR L1 and a heavy chain variable domain with one or more alterations in loop H1 and/or in CDR H3.

According to one aspect of the invention, the antibody variant or antibody variable domain has one or more substitutions according to the invention herein at one or more of amino acid positions 26L, 27L, 28L, 30L, 31L, 32L, 49L, 50L, 52L, 53L, 54L, 56L, 93L or 94L of a light chain variable domain of the antibody and/or at one or more of amino acid positions 25H, 28H, 30H, 54H, 56H, 61H, 62H, 64H, 97H, 98H, 99H and/or 100aH of a heavy chain variable domain of the antibody. Moreover, substitutions at these positions can be combined. For instance, substitutions at two, three or four of amino acid positions 26L, 27L, 28L or 30L of a light chain variable domain of the antibody may be combined. One may combine a modified heavy chain variable domain (e.g. with substitutions at positions 28H and/or 100aH) with the modified light chain variable domain (e.g. with substitutions at positions 26L, 27L, 28L and/or 30L). The residue numbering here is according to Kabat et al., Sequences of Proteins of Immunological Interest, 5th Ed. Public Health Service, National Institutes of Health, Bethesda, Md. (1991).

The invention also provides an antibody variant or modified antibody variable domain obtainable according to the method of described herein. Preferably, the antibody variant or modified antibody variable domain comprises amino acid alteration(s) in or adjacent to hypervariable region(s) thereof which increase charge complementarity between the antibody variant and an antigen to which it binds. Examples of such modified variable domains include a light chain variable domain comprising a CDR L1 sequence selected from SATKKIKNYLN (SEQ ID NO:6) or SATKKITNYLN (SEQ ID NO:7), e.g. a light chain variable domain comprising the amino acid sequence of SEQ ID NO:3 or SEQ ID NO:4; and a heavy chain variable domain comprising the substitutions of T28D and S100aR, e.g., the amino acid sequence of SEQ ID NO:5, SEQ ID NO:8, SEQ ID NO:9 or SEQ ID NO:10. Optionally, these light and heavy chain variable domain sequences are combined in an antibody variant, e.g. one comprising the light chain variable domain sequence of SEQ ID NOS:4 and the heavy chain variable domain sequence selected from SEQ ID NO:5, 8, 9 or 10. Preferably, the antibody variant comprises the CDR L1 sequence of SEQ ID NO:7 in its light chain variable domain and the (T28D,S100aR) substitution in its heavy chain variable domain, such combination of substitutions is referred to as the “34-TKKT” variant in the Example herein. Such substitutions (V_{H}-(T28,S100aR)+V_{L}-(S26T,Q27K,D28K,S30T)) can be made in various parent antibodies, including but not limited to the anti-VEGF antibody selected from the group consisting of Y0101, Y0317, humanized anti-VEGF F(ab)-12, Y0192, Y0238-3, Y0239-19, Y0313-2, and VNERK mutant. For example, a “34-TKKT+VNERK+H97Y” variant is generated by combining alterations of the “34-TKKT”, the “H97Y” and the VNERK variants (SEQ ID NOS:4 and 8 for light and heavy chain variable domains, respectively).

Nucleic acid molecules encoding amino acid sequence variants are prepared by a variety of methods known in the art. These methods include, but are not limited to, oligonucleotide-mediated (or site-directed) mutagenesis, PCR mutagenesis, and cassette mutagenesis of an earlier prepared variant or a non-variant version of the parent antibody. The preferred method for making variants is site directed mutagenesis (see, e.g., Kunkel, Proc. Natl. Acad. Sci. USA 82:488 (1985)). Moreover, a nucleic acid sequence can be made synthetically, once the desired amino acid sequence is arrived at conceptually. One can also make the antibody variant by peptide synthesis, peptide ligation or other methods.

Following production of the antibody variant, the activity of that molecule relative to the parent antibody may be determined. As noted above, this may involve determining the association rate, and/or binding affinity, and/or other biological activities of the antibody. In a preferred embodiment of the invention, a panel of antibody variants are prepared and are screened for association rate and/or binding affinity for the antigen and/or potency in one or more assays. One or more of the antibody variants selected from an initial screen is/are optionally subjected to one or more further functional assays to confirm that the antibody variant(s) have improved activity in more than one assay.

Aside from the above alteration(s) in hypervariable region(s) of the parent antibody, one may make other alterations in the amino acid sequences of one or more of the hypervariable regions. For example, the above amino acid alterations may be combined with deletions, insertions or substitutions of other hypervariable region residues. Moreover, one or more alterations (e.g. substitutions) of FR residues may be introduced in the parent antibody where these result in an improvement in the binding affinity of the antibody variant for the antigen. Examples of framework region residues to modify include those which non-covalently bind antigen directly (Amit et al. Science 233:747-753 (1986)); interact with/effect the conformation of a CDR (Chothia et al. J. Mol. Biol. 196:901-917 (1987)); and/or participate in the V_{L}-V_{H} interface (EP 239 400B1). Such amino acid sequence alterations may be present in the parent antibody, may be made simultaneously with the amino acid insertion(s) herein, or may be made after a variant with an amino acid alteration(s) according to the invention herein is generated. Alterations in constant domain sequence(s) of the parent antibody or antibody variant are also contemplated herein, e.g. those which improve, or diminish, antibody effector function(s). See, e.g., U.S. Pat. No. 6,194,551B1; WO 99/51642; Idusogie et al. J. Immunol. 164: 4178-4184 (2000); WO00/42072 (Presta); and Shields et al. J. Biol. Chem. 9(2): 6591-6604 (2001), expressly incorporated herein by reference.

The antibody variants may be subjected to other modifications, oftentimes depending on the intended use of the antibody. Such modifications may involve further alteration of the amino acid sequence, fusion to heterologous polypeptide(s) and/or covalent modification. With respect to amino acid sequence alterations, exemplary modifications are elaborated above. For example, any cysteine residue not involved in maintaining the proper conformation of the antibody variant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid variant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Techniques for producing antibodies, which may be the parent antibody and therefore require modification according to the techniques elaborated herein, follow:

A. Antibody Preparation

(i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable domain sequences. The human sequence which is closest to that of the rodent is then accepted as the human FR for the humanized antibody (Sims et al., J. Immunol., 151:2296 (1993); Chothia et al., J. Mol. Biol., 196:901 (1987)). Another method uses a “consensus” framework based on a particular subgroup of human antibody sequences. The same consensus framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as improved affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

### (vi) Multispecific Antibodies

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein. Examples of EsAbs include those with one arm directed against a tumor cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185, anti-CD3/anti-p97, anti-CD3/anti-renal cell carcinoma, anti-CD3/anti-QVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell ahesion molecule (NCAM)/anti-CD3, anti-folate binding protein (FBP)/anti-CD3, anti-pan carcinoma associated antigen (AMOC-31)/anti-CD3; BsAbs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA); BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor:CD3 complex/anti-influenza, anti-FcγR/anti-HIV; BsAbs for tumor detection in vitro or in vivo such as anti-CEA/anti-EOTUBE, anti-CEA/anti-DPTA, anti-p185^{HER2}/anti-hapten; BsAbs as vaccine adjuvants; and BsAbs as diagnostic tools such as anti-rabbit IgG/anti-ferritin, anti-horse radish peroxidase (HRP)/anti-hormone, anti-somatostatin/anti-substance P, anti-HRP/anti-FITC. Examples of trispecific antibodies include anti-CD3/anti-CD4/anti-CD37, anti-CD3/anti-CD5/anti-CD37 and anti-CD3/anti-CD8/anti-CD37. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab, portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

(vii) Exemplary Antibodies

Preferred antibodies within the scope of the present invention include anti-HER2 antibodies including rhuMAb 4D5 (HERCEPTIN®) (Carter et al., Proc. Natl. Acad. Sci. USA, 89:4285-4289 (1992), U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108, B1 or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN™ (Kim et al., Growth Factors, 7:53-64 (1992), International Publication No. WO 96/30046, and WO 98/45331, published Oct. 15, 1998); anti-Tissue Factor (TF) antibodies (European Patent No. 0420937B1 granted Nov. 9, 1994) including humanized and/or affinity matured anti-VEGF antibodies such as D3H44 (WO01/70984); anti-PSCA antibodies (WO 01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58:377-380 (1994)); anti-CD18 (U.S. Pat. No. 5,622,700, issued Apr. 22, 1997, or as in WO 97/26912, published Jul. 31, 1997); anti-IgE (U.S. Pat. No. 5,714,338, issued Feb. 3, 1998 or U.S. Pat. No. 5,091,313, issued Feb. 25, 1992, WO 93/04173 published Mar. 4, 1993, International Application No. PCT/US98/13410 filed Jun. 30, 1998, U.S. Pat. No. 5,714,338, Presta et al., J. Immunol. 151:2623-2632 (1993), and International Publication No. WO 95/19181)); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al. J. Immunol. 156(4):1646-1653 (1996), and Dhainaut et al. Crit. Care Med. 23(9):1461-1469 (1995)); anti-human α₄β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al. Arthritis Rheum 39(1):52-56 (1996)); anti-CD52 antibodies such as CAMPATH-1H (Riechmann et al. Nature 332:323-337 (1988); anti-Fc receptor antibodies such as the M22 antibody directed against FcγRI as in Graziano et al. J. Immunol. 155(10):4996-5002 (1995); anti-carcinoembryonic antigen (CEA) antibodies such as hMN-14 (Sharkey et al. Cancer Res. 55(23Suppl): 5935s-5945s (1995)); antibodies directed against breast epithelial cells including huBrE-3, hu-Mc 3 and CHL6 (Ceriani et al. Cancer Res. 55(23): 5852s-5856s (1995); and Richman et al. Cancer Res. 55(23 Supp): 5916s-5920s (1995)); antibodies that bind to colon carcinoma cells such as C242 (Litton et al. Eur J. Immunol. 26(1):1-9 (1996)); anti-CD38 antibodies, e.g. AT 13/5 (Ellis et al. J. Immunol. 155(2):925-937 (1995)); anti-CD33 antibodies such as Hu M195 (Jurcic et al. Cancer Res 55(23 Suppl):5908s-5910s (1995)) and CMA-676 or CDP771; anti-CD22 antibodies such as LL2 or LymphoCide (Juweid et al. Cancer Res 55(23 Suppl):5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-avp3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 or the anti-HLA DR antibody Oncolym (Lym-1).

(viii) Immunoconjugates

The invention also pertains to immunoconjugates comprising the antibody described herein conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate).

Chemotherapeutic agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugate antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

B. Vectors, Host Cells and Recombinant Methods

The invention also provides isolated nucleic acid encoding an antibody variant as disclosed herein, vectors and host cells comprising the nucleic acid, and recombinant techniques for the production of the antibody variant.

For recombinant production of the antibody variant, the nucleic acid encoding it may be isolated and inserted into a replicable vector for further cloning (amplification of the DNA) or for expression. DNA encoding the antibody variant is readily isolated and sequenced using conventional procedures (e.g., by using oligonucleotide probes that are capable of binding specifically to genes encoding the heavy and light chains of the antibody variant). Many vectors are available. The vector components generally include, but are not limited to, one or more of the following: a signal sequence, an origin of replication, one or more marker genes, an enhancer element, a promoter, and a transcription termination sequence. Such vector components are described in WO00/29584, expressly incorporated herein by reference.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody variant of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody variant can be produced intracellularly, in the periplasmic space, or directly secreted into the medium. If the antibody variant is produced intracellularly, as a first step, the particulate debris, either host cells or lysed fragments, is removed, for example, by centrifugation or ultrafiltration. Where the antibody variant is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody variant. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody variant comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody variant to be recovered.

C. Pharmaceutical Formulations

Therapeutic formulations of the antibody variant are prepared for storage by mixing the antibody variant having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody variant, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

D. Non-Therapeutic Uses for the Antibody Variant

The antibody variants of the invention may be used as affinity purification agents. In this process, the antibodies are immobilized on a solid phase such a Sephadex resin or filter paper, using methods well known in the art. The immobilized antibody variant is contacted with a sample containing the antigen to be purified, and thereafter the support is washed with a suitable solvent that will remove substantially all the material in the sample except the antigen to be purified, which is bound to the immobilized antibody variant. Finally, the support is washed with another suitable solvent, such as glycine buffer, pH 5.0, that will release the antigen from the antibody variant.

The variant antibodies may also be useful in diagnostic assays, e.g., for detecting expression of an antigen of interest in specific cells, tissues, or serum.

For diagnostic applications, the antibody variant typically will be labeled with a detectable moiety. Numerous labels are available which can be generally grouped into the following categories:

(a) Radioisotopes, such as ³⁵S, ¹⁴C, ¹²⁵I, ³H, and ¹³¹ I. The antibody variant can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) beta-D-galactosidase (beta-D-Gal) with a chromogenic substrate (e.g., p-nitrophenyl-beta-D-galactosidase) or fluorogenic substrate 4-methylumbelliferyl-beta-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody variant. The skilled artisan will be aware of various techniques for achieving this. For example, the antibody variant can be conjugated with biotin and any of the three broad categories of labels mentioned above can be conjugated with avidin, or vice versa. Biotin binds selectively to avidin and thus, the label can be conjugated with the antibody variant in this indirect manner. Alternatively, to achieve indirect conjugation of the label with the antibody variant, the antibody variant is conjugated with a small hapten (e.g., digoxin) and one of the different types of labels mentioned above is conjugated with an anti-hapten antibody variant (e.g., anti-digoxin antibody). Thus, indirect conjugation of the label with the antibody variant can be achieved.

In another embodiment of the invention, the antibody variant need not be labeled, and the presence thereof can be detected using a labeled antibody which binds to the antibody variant.

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody variant. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyze is bound by a first antibody which is immobilized on a solid support, and thereafter a second antibody binds to the analyze, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tumor sample may be fresh or frozen or may be embedded in paraffin and fixed with a preservative such as formalin, for example.

The antibodies may also be used for in vivo diagnostic assays. Generally, the antibody variant is labeled with a radionuclide (such as ¹¹¹In, ⁹⁹Tc, ¹⁴C, ¹³¹I, ¹²⁵I, ³H, ³²P or ³⁵S) so that the tumor can be localized using immunoscintiography.

E. Diagnostic Kits

As a matter of convenience, the antibody variant of the present invention can be provided in a kit, i.e., a packaged combination of reagents in predetermined amounts with instructions for performing the diagnostic assay. Where the antibody variant is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

F. In Vivo Uses for the Antibody Variant

For therapeutic applications, the antibody variants of the invention are administered to a mammal, preferably a human, in a pharmaceutically acceptable dosage form such as those discussed above, including those that may be administered to a human intravenously as a bolus or by continuous infusion over a period of time, by intramuscular, intraperitoneal, intra-cerebrospinal, subcutaneous, intra-articular, intrasynovial, intrathecal, oral, topical, or inhalation routes. The antibodies also are suitably administered by intra-tumoral, peri-tumoral, intra-lesional, or peri-lesional routes, to exert local as well as systemic therapeutic effects. The intra-peritoneal route is expected to be particularly useful, for example, in the treatment of ovarian tumors. In addition, the antibody variant is suitably administered by pulse infusion, particularly with declining doses of the antibody variant. Preferably the dosing is given by injections, most preferably intravenous or subcutaneous injections, depending in part on whether the administration is brief or chronic.

For the prevention or treatment of disease, the appropriate dosage of antibody variant will depend on the type of disease to be treated, the severity and course of the disease, whether the antibody variant is administered for preventive or therapeutic purposes, previous therapy, the patient's clinical history and response to the antibody variant, and the discretion of the attending physician. The antibody variant is suitably administered to the patient at one time or over a series of treatments.

The example herein concerns an anti-VEGF antibody. Anti-VEGF antibodies are useful in the treatment of various neoplastic and non-neoplastic diseases and disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Non-neoplastic conditions that are amenable to treatment include rheumatoid arthritis, psoriasis, atherosclerosis, diabetic and other proliferative retinopathies including retinopathy of prematurity, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the VEGF antibodies of the present invention are expected to be especially useful in reducing the severity of AMD.

Depending on the type and severity of the disease, about 1 μg/kg to 15 mg/kg (e.g., 0.1-20 mg/kg) of antibody variant is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. Due to the improved association rate of the antibody variant, it is contemplated that lower doses of the antibody variant (compared to the parent antibody) may be administered.

The antibody variant composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody variant to be administered will be governed by such considerations, and is the minimum amount necessary to prevent, ameliorate, or treat a disease or disorder. The antibody variant need not be, but is optionally formulated with one or more agents currently used to prevent or treat the disorder in question. The effective amount of such other agents depends on the amount of antibody variant present in the formulation, the type of disorder or treatment, and other factors discussed above. These are generally used in the same dosages and with administration routes as used hereinbefore or about from 1 to 99% of the heretofore employed dosages.

G. Articles of Manufacture

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody variant. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

H. Antigen Association Rate Assay

The present application also describes an assay method which can be used to measure antigen association rate of an antibody (e.g. an antibody variant such as those described herein). The method is particularly adapted for antibodies with slow association rates (e.g. those with an association constant for antigen slower than about 10⁵ M⁻¹ sec⁻¹, or slower than about 10⁶ M⁻¹ sec⁻¹) such that formation of the antibody-antigen complex can be quantified over time. One example of an antibody with a slow antigen association constant is an anti-VEGF antibody which binds VEGF, exemplified by the various anti-VEGF antibodies referenced herein.

The assay method herein comprises: (1) combining antibody and antigen in solution, and then; (2) determining formation of antibody-antigen complex over time. Hence, measurement of complex formation occurs after the antibody and antigen have been combined. Formation of the complex over time can be determined using various methods such as determining fluorescence or adsorption of the complex, or using NMR. However, according to the preferred embodiment, the second step of the method comprises measuring fluorescence emission intensity of the antibody-antigen complex over time. This may be achieved where the antibody or antigen comprises a tryptophan residue at the antigen-antibody binding interface, so that one can measure fluorescence emission intensity of the tryptophan residue (which changes when the tryptophan residue is buried at the binding interface). Fluorescence emission intensity may be determined using an excitation wavelength from about 280-310 nm (e.g. 295 nm) and detecting emission at a wavelength from about 330-360 nm (e.g. about 340 nm).

The following examples are intended merely to illustrate the practice of the present invention and are not provided by way of limitation. The disclosures of all patent and scientific literatures cited herein are expressly incorporated in their entirety by reference.

### Example 1

The present example demonstrates that the principles of electrostatic steering can be applied to increase the on-rate of an antibody's binding to its antigen, without extensive calculations, by identifying potential on-rate amplification sites through a series of criteria that reduce the list of target sites to an experimentally tractable number. A particular example is the modification of the anti-VEGF Y0101 antibody Fab fragment (FIGS. 1A-B). Fab Y0101 with mutations made at identified target sites, characterized by a fluorescence-based assay, showed association rates improved by nearly an order of magnitude. Furthermore, the association rates observed for the Fab-VEGF complex showed no correlation with those predicted by calculation of the Debye-Huckel energy of interaction. The variants of Fab Y0101 with faster on-rates are expected to be more potent antagonists of VEGF due to their higher affinity, but also more efficacious due to faster binding. This importance of the latter should not be understated, as the association and dissociation rates of the Fab Y0101-VEGF complex are orders of magnitude slower than typical protein-protein interactions (Chen et al. Journal of Molecular Biology 293(4): 865-81 (1999); Gabdoulline et al. Journal of Molecular Biology 306(5): 1139-55 (2001)). The criteria described herein for the identification of ON-RAMPS is sufficient for guiding the redesign of an antibody fragment for improved association and overall binding affinity with its antigen.

### Materials and Methods

### Identification of On-Rate Amplification Sites (On-RAMPS)

As there are about 445 residues in an antibody fragment (Fab), one step in improving its association rate with ligand involves identification of residues, which when mutated to increase charge complementarity, will significantly alter the electrostatic interaction energy between the two proteins. The following criteria were applied to identify these “On-Rate Amplification Sites” (On-RAMPS).

1) The residue had at least one third of its side chain surface area exposed to solvent, as mutation of buried residues may destabilize the Fab.
2) The residue was within at least 16 Å of VEGF in the bound state, as electrostatic attractive forces may decay as a function of distance.
3) The residue did not directly contact VEGF in the bound state, as mutation of direct contact residues may destabilize the bound complex.
4) Preference was given to those residues that were within the complementarity determining regions (CDRs) over those that were not, as there are indications that they are less likely to induce immunogenic responses in patients.
5) Only those residues for which it was possible to increase the charge complementarity between the Fab and the antigen were considered. For example, V_{L}-D28 of Y0101 can be mutated to either neutralize (D28N) or reverse (D28K) its charge to better complement the negatively charged antigen, whereas residue V_{H}-K64 cannot be mutated to increase its positive charge.

### Mutagenesis, Protein Expression and Purification

The short isoform of VEGF (8-109) was produced as described previously (Christinger et al. Proteins 26(3): 353-7 (1996)). The method for constructing and purifying mutant variants of the Fab has been described previously (Muller et al. Structure 6(9): 1153-67 (1998)). Briefly, point mutations were made by oligonucleotide directed mutagenesis by the methods developed by Kunkel (Kunkel, T. A. Proceedings of the National Academy of Sciences of the USA 82(2): 488-92 (1985)). Fab is expressed upon induction in the non-suppressor E. coli cell line 34B8 (Baca et al. Journal of Biological Chemistry 272(16): 10678-84 (1997)) and purified by affinity chromatography with protein G resin (Amersham) after osmostic shock of harvested cells. Typical yields are 2 nmoles Fab per 1 liter growth.

### Association Rate Assay

In the experiments described here, the fluorescence emission intensity (λ_{excitation}=295 nm; λₑₘᵢₛₛᵢₒₙ=340 nm, 16 nm bandpass) was measured using an 8000-series SLM-Aminco spectrophotometer (THERMOSPECTRONIC®) as VEGF was added to a stirred cuvette containing approximately 10 nM Fab in 25 mM Tris, pH 7.2, held at 37° C.

### Dissociation Rate Assay

Dissociation rates were measured by surface plasmon resonance on a BIACORE-2000® instrument (BIAcore, Inc.) as described previously (Muller et al. Structure 6(9): 1153-67 (1998)). VEGF was immobilized by amine coupling to a B1 chip at approximately 10 resonance-response units. Fab binding was measured at 1 μM, 500 nM, 250 nM, 125 nM, 62.5 nM, and 31.3 nM. Dissociation was calculated assuming a one-to-one binding model. All experiments were performed at 37° C. in phosphate buffered saline solution, pH 7.2, containing 0.05% Tween-20, 0.01% NaN₃ and at a flow rate of 20 μL min⁻¹.

### Results

### Development of the Association Rate Assay

While surface plasmon resonance technology has been demonstrated to be suitable for affinity measurements, subtle differences among variants of a particular binding interaction may go unnoticed for multiple reasons, ranging from the complexities of flow-dynamics (Fivash et al. Current Opinion in Biotechnology 9(1): 97-101 (1998)) and non-specific amine coupling (Kortt et al. Analytical Biochemistry 253(1): 103-11 (1997)) to a simple inability to accurately determine the concentrations of properly folded and active proteins.

Since the work presented here is concerned with differences in binding rates among variants of the anti-VEGF Fab, an assay was developed that was sensitive enough to detect subtle differences in on-rates, representative of the interaction in solution, and independent of the concentration of various Fab variants.

The fluorescence properties of tryptophan residues are sensitive to their local environment (Lakowicz, J. R. (1999). Principles of fluorescence spectroscopy. 2nd edit, Kluwer Academic Press, New York, N.Y.). As revealed by the co-structure of VEGF and the anti-VEGF Fab used in this study (Muller et al. Structure 6(9): 1153-67 (1998)), there are three tryptophans in the Fab that form direct contact with VEGF in the bound state and whose fluorescence properties may be expected to change upon going from an unbound to a bound state. There are no tryptophans in VEGF, but there are two tyrosines and one phenylalanine that form the binding interface with the Fab (Muller et al. Structure 6(9): 1153-67 (1998)) that may contribute to the fluorescence spectrum if excited. To circumvent this potential source of error, the fluorescence assay is performed with an excitation wavelength of 295 nm, which minimally overlaps the excitation spectra of tyrosine and phenylalanine (Lakowicz, J. R. Principles of fluorescence spectroscopy. 2nd edit, Kluwer Academic Press, New York, N.Y. (1999)).

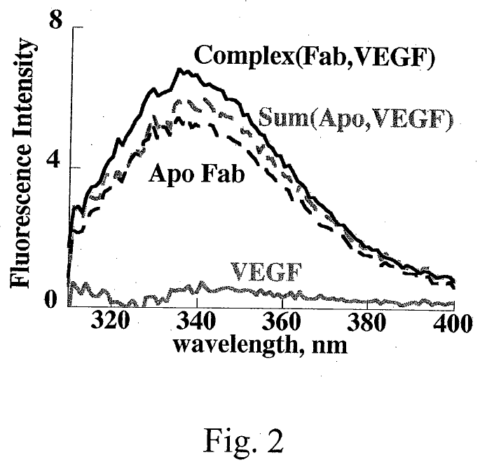

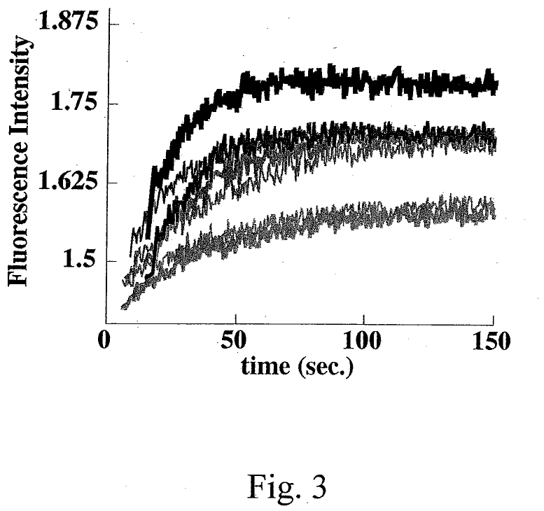

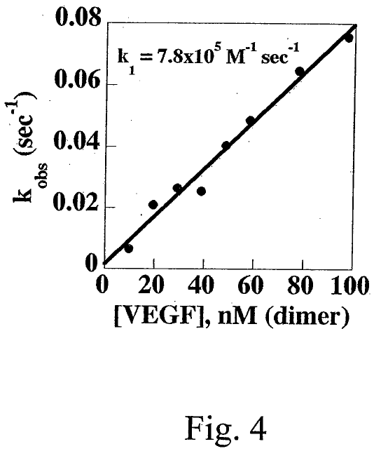

The fluorescence intensity of the Fab-VEGF complex is greater than the sum of the individual fluorescence intensities of the components (FIG. 2). The rate of increase of the fluorescence intensity can be fit to a single exponential curve (FIG. 3). Plotting the observed rate as a function of VEGF concentration permits pseudo-first-order analysis, the slope being k₁ for the reaction, the y-intercept being k₋₁ (FIG. 4) (Johnson, K. A. Transient-state kinetic analysis of enzyme reaction pathways. In The Enzymes, Vol. 20: pp. 1-61. Academic Press, Inc. (1992)).

### Identification of On-RAMPS

By applying the criteria outlined above, the number of potential sites for mutagenesis was reduced from 445 residues to 22 (Table 1). The first criterion, being solvent exposed, reduces the number to 173. The second, being within 16 Å of VEGF, reduces the number to 47. The third, not directly contacting VEGF, reduces the number to 31. The fourth, that the residue lie within the CDRs, reduces that to 23. Finally, one additional residue (V_{H}-K64) is eliminated by the final criterion, as its complementarity with the negatively charged VEGF cannot be increased. The predicted on-rate for mutation of each of these residues to a positively charged residue was calculated according the method of Schreiber et al. (2000) Nat. Struct. Biol. 7:537-41.

TABLE 1
Potential ON-RAMPS of Fab Y0101

| | | minimum | |
|---|---|---|---|
| | | distance from | Calculated on-rate |
| Residue | % SASA | VEGF (Å) | (relative to wt.) |
| Light Chain | | | |
| Ser 26 | 38 | 15.7 | 1.2 |
| Gln 27 | 58 | 11.8 | 1.1 |
| Asp 28 | 66 | 13.4 | 1.4 |
| Ser 30 | 50 | 11.2 | 1.3 |
| Asn 31 | 44 | 12.5 | 1.2 |
| Tyr 32 | 48 | 6.3 | 1.1 |
| Phe 50 | 43 | 9.7 | 1.2 |
| Ser 52 | 60 | 15.3 | 1.2 |
| Ser 53 | 42 | 10.4 | 1.2 |
| Leu 54 | 37 | 13.9 | 1.1 |
| Ser 56 | 90 | 10.5 | 1.1 |
| Thr 93 | 56 | 6.9 | 1.1 |
| Val 94 | 34 | 3.9 | 1.1 |
| Heavy Chain | | | |
| Ser 25 | 54 | 15.1 | 1.1 |
| Thr 28 | 69 | 6.4 | 1.1 |
| Thr 30 | 36 | 5.9 | 1.2 |
| Thr 54 | 36 | 4.4 | 1.2 |
| Glu 56 | 80 | 6.5 | 1.6 |
| Ala 61 | 93 | 11.4 | 1.1 |
| Asp 62 | 87 | 15.3 | 1.1 |
| Tyr 99 | 84 | 3.5 | 1.6 |
| Ser 100a | 68 | 4.9 | 1.3 |

Residues are numbered according the Kabat system (Kabat et al. Sequences of Proteins of Immunological Interest, 5th Edition., National Institute of Health, Bethesda, Md. (1991)). % SASA calculated using a 1.4 Å probe radius.

### Observed Association Rates

Since the net formal charge on VEGF is −10 (calculated by assigning +1 to the N-termini, lysines, and arginines, and −1 to the C-termini, aspartates and glutamates), mutations were made to increase the net positive charge on the Fab (wild-type=+2) at the periphery of the binding interface. Mutation of these residues results in increases in association rate as great as two fold relative to Y0101 (Table 2). On the other hand, mutations of residues that are solvent exposed, but not within 16 Å of VEGF (Table 2, unqualified), show little change, thus illustrating the utility of the ON-RAMPS criteria. Further increases in the association rate of the anti-VEGF Fab were achieved by mutating multiple residues (Table 3), with the fastest binding variant, “34-TKKT” (V_{H}-(T28D, S100aR)+V_{L}-(S26T, Q27K, D28K, S30T)) having an association rate 6-fold higher than that of Y0101. Conversely, mutations that gave rise to charge repulsion resulted in decreased association rates (Table 3: mutant V_{L}-S26E, Q27E, D28E, S30E and mutant V_{L}-T51E, S52E, S53E, L54E).

TABLE 2
Binding Constants of single mutations

| | k₁ | k₋₁ | k₋₁ | |
|---|---|---|---|---|
| | (×10⁵ M⁻¹ | (×10⁻⁴ sec⁻¹) | (×10⁻⁴ sec⁻¹) | K_{d} |
| Mutation | sec⁻¹) | 0 M NaCl | 0.15 M NaCl | (×10⁻⁹ M) |
| Y0101 | 5.4 ± 0.3 | 3.9 ± 1.1 | 1.3 ± 0.5 | 0.7 |
| (wild-type) | | | | |
| Light Chain | | | | |
| R18Q* | 4.8 | 3.8 ± 0.5 | 0.9 ± 0.3 | 0.8 |
| R18E* | 5.2 | 2.6 ± 0.7 | 0.9 ± 0.2 | 0.5 |
| S26K | 7.4 | 3.3 ± 0.5 | 0.6 ± 0.3 | 0.4 |
| Q27K | 6.7 | 4.0 ± 0.4 | 0.7 ± 0.4 | 0.6 |
| D28K | 7.0 | 3.3 ± 0.2 | 0.9 ± 0.4 | 0.5 |
| D28N | 6.5 | 2.8 ± 0.8 | 0.9 ± 0.3 | 0.4 |
| S30K | 9.7 | 3.3 ± 0.5 | 0.3 ± 0.1 | 0.3 |
| S30N | 5.5 | 3.3 ± 0.4 | 0.9 ± 0.3 | 0.6 |
| N31K | 6.9 | 3.3 ± 0.5 | 0.3 ± 0.2 | 0.5 |
| N31R | 7.8 | 3.8 ± 0.2 | 0.4 ± 0.2 | 0.5 |
| Y32K | 7.3 | 2.5 ± 0.4 | 0.4 ± 0.2 | 0.3 |
| Y32R | 7.1 | LE | LE | — |
| S52K | 6.2 | 3.8 ± 0.2 | 0.6 ± 0.2 | 0.6 |
| S53K | 8.0 | 3.8 ± 0.2 | 0.5 ± 0.2 | 0.5 |
| L54K | 4.4 | LE | LE | — |
| V94E | 1.3 | 10.1 ± 1.0 | 4.4 ± 0.8 | 7.8 |
| E195R* | 4.9 | BG | LE | — |
| E195Q* | 5.9 | 5.3 ± 2.5 | 1.5 ± 0.7 | 0.9 |
| E195L* | 7.3 | BG | 0.6 ± 0.2 | — |
| Heavy Chain | | | | |
| T28K | 3.6 | LE | LE | — |
| T28R | 4.0 | 4.8 ± 0.4 | LE | 1.2 |
| T28E | 7.8 | 4.8 ± 0.3 | 1.0 ± 0.1 | 0.6 |
| T28D | 10. | 2.9 ± 0.2 | 0.8 ± 0.3 | 0.3 |
| T30D | 6.0 | 2.6 ± 1.4 | 1.0 ± 0.1 | 0.4 |
| T30E | 4.8 | 7.2 ± 0.3 | 1.4 ± 0.2 | 1.5 |
| E56K | 4.8 | 4.4 ± 0.3 | LE | 0.9 |
| Y99K | 3.8 | 1.5 ± 1.2 | 1.4 ± 0.3 | 0.4 |
| Y99R | 6.0 | 1.4 ± 1.3 | 1.6 ± 0.3 | 0.2 |
| S100aR | 8.7 | 2.4 ± 0.8 | 0.7 ± 0.1 | 0.3 |
| D218N* | 4.9 | BG | 0.8 ± 0.2 | — |
| D218K* | 5.3 | BG | 0.6 ± 0.4 | — |

Residues are numbered according the Kabat system (Kabat et al. Sequences of Proteins of Immunological Interest, 5th Edition., National Institute of Health, Bethesda, Md. (1991)). k₁ determined by the fluorescence-based assay (+standard deviation of three experiments, wild-type only). k₋₁ determined by surface plasmon resonance (±standard deviation of 12 experiments). k₁ and k₋₁ (0 M NaCl) experiments were performed in 25 mM Tris, pH 7.2, at 37° C. k₋₁ (0.15 M NaCl) experiments performed in 25 mM Tris, 150 mM NaCl, at 25° C. K_{d} is calculated from 0 M NaCl data. *, residues that do not meet the ON-RAMPS criteria; LE, low expression of Fab limited analysis; BG, background binding to control flow cell limited analysis of SPR data.

TABLE 3
Binding constants of multiple mutations

| | k₁ | k₋₁ | k₋₁ | |
|---|---|---|---|---|
| | (×10⁵ M⁻¹ | (×10⁻⁴ sec⁻¹) | (×10⁻⁴ sec⁻¹) | K_{d} |
| Mutations | sec⁻¹) | 0 M NaCl | 0.15 M NaCl | (×10⁻⁹ M) |
| Light Chain | | | | |
| S26E, Q27E, D28E, S30E | 2.6 | 5.1 ± 0.8 | 0.8 ± 0.2 | 2.0 |
| S26K, Q27K, D28N, S30T | 6.1 | BG | LE | — |
| S26K, D28K, S30K | 13 | LE | LE | — |
| S26K, Q27K, D28N, S30K | 13 | BG | 0.7 ± 0.1 | — |
| S26K, Q27K, D28K, S30T | 21 | BG | 0.4 ± 0.1 | — |
| S26T, Q27K, D28K, S30K | 25 ± 1.0 | BG | 0.6 ± 0.1 | — |
| S26T, Q27K, D28K, S30T | 29 ± 2.9 | BG | 0.6 ± 0.1 | — |
| T51E, S52E, S53E, L54E | 3.3 | 4.4 ± 1.0 | 40.8 ± 14.7 | 1.3 |
| S52K, S53K, L54T | 13 | BG | LE | — |
| S26K, Q27K, D28K, S30K, | 24 | 7.8 ± 1.1 | 0.9 ± 0.2 | 0.3 |
| T51K, S52K, S53K, L54K | | | | |
| Heavy Chain | | | | |
| T28D, S100aR | 14 | BG | 1.1 ± 0.3 | — |
| Fastest Binding Variant | | | | |
| 34-TKKT | 33 ± 3.9 | 2.6 ± 1.2 | 0.2 ± 0.1 | 0.08 |

Residues are numbered according the Kabat system (Kabat et al. Sequences of Proteins of Immunological Interest, 5th Edition., National Institute of Health, Bethesda, Md. (1991)). k₁ determined by the fluorescence-based assay (±standard deviation of three experiments). k₋₁ determined by surface plasmon resonance (+standard deviation of 12 experiments). k₁ and k₋₁ (0 M NaCl) experiments were performed in 25 mM Tris, pH 7.2, at 37° C. k₋₁ (0.15 M NaCl) experiments performed in 25 mM Tris, 150 mM NaCl, at 25° C. K_{d} is calculated from 0 M NaCl data. 34-TKKT, mutant V_{H}-(T28, S100aR)+V_{L}-(S26T,Q27K,D28K,S30T). LE, low expression of Fab limited analysis; BG, background binding to control flow cell limited analysis of SPR data.

### Comparison of Observed and Calculated Association Rates

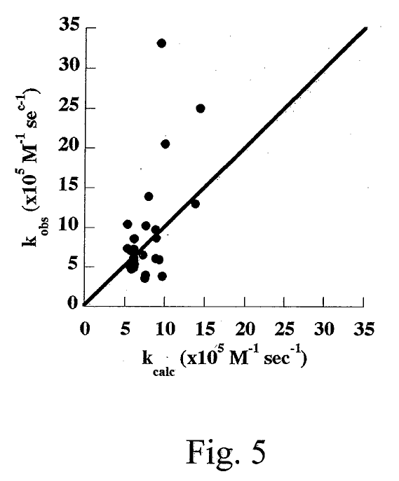

It has been suggested that calculation of the Debye-Huckel energy of electrostatic interaction is a powerful and accurate predictor of association rate (Selzer, T. & Schreiber, G. Journal of Molecular Biology 287(2): 409-19 (1999)). The program used by Selzer and Schreiber is available for public use at their internet address (http://www.weizmann.ac.il/home/bcges/PARE.html). Using this program and following their guidelines, the association rates of the different variants constructed in this work were calculated for comparison with experimentally determined values. The plot of k_{obs} against k_{calc} indicates poor correlation, with an R value of 0.46 (FIG. 5).

### Salt Dependence of Association Rates

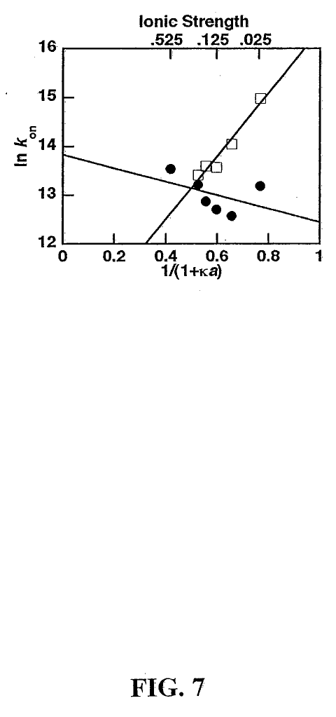

To illustrate that the difference in association rates between variants is attributable to the electrostatic interaction between the Fabs and VEGF, rather than a general structural rearrangement of the binding interface, we measured the association rates of the wild-type Fab Y0101 and 34-TKKT at different salt concentrations (FIG. 7). The difference in association rate between the fastest binding variant and Y0101 in 150 mM NaCl is less than 2-fold.

Importantly, the electrostatic energy of interaction between the Fab and VEGF as calculated from the structure of the complex (Y0101=0.28 kcal mol⁻¹, 34-TKKT=−1.07 kcal mol⁻¹) is of the correct sign (though differing in magnitude) with the value determined from the slope of FIG. 7. (Y0101=0.86 kcal mol⁻¹, 34-TKKT=−4.0 kcal mol⁻¹).

Combination of Fast On-Rate Variants with Other Affinity Matured Variants

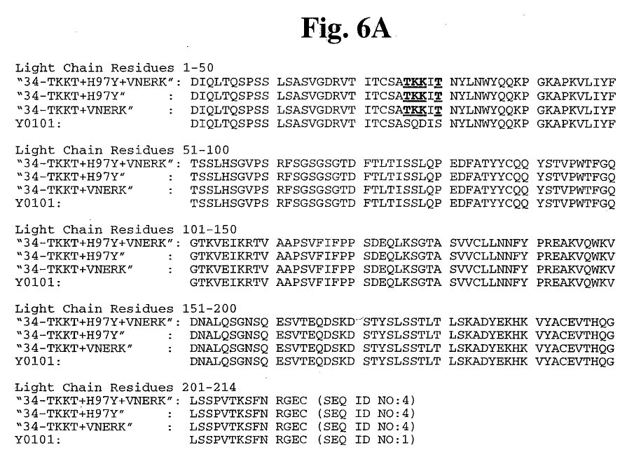

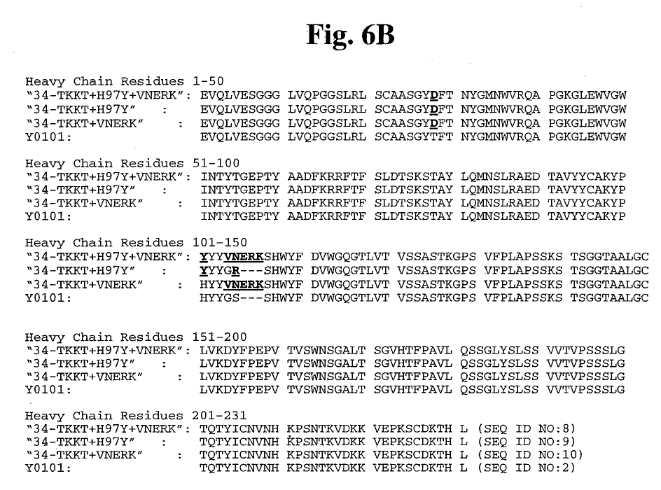

The fast on-rate variants described above can be combined with other identified variants to achieve even greater binding affinities. For example, the fastest binding variant “34-TKKT” can be combined with known anti-VEGF variants such as Fab-12, VNERK or Y0317. Additional sequence alterations can be made to further optimize binding affinity as well as other physical or chemical properties of the molecule. FIGS. 6A and 6B provide alignments of three such “combination” variants, in which the substitutions of the “34-TKKT” are made together with either the VNERK insertion, or the H97Y substitution, or both the VNERK insertion and the H97Y substitution. The resulting variants are expected to possess greater binding affinities to VEGF and hence better efficacy when used as therapeutic antagonists to VEGF.

### Example 2

The principles of identifying On-RAMPS and generating faster on-rate variants described above, in the context of anti-VEGF antibodies, can be similarly applied to other antibody variants as well, including but not limited to anti-TF and anti-HER2 antibody variants.

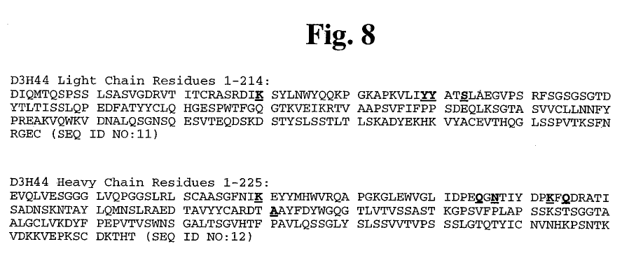

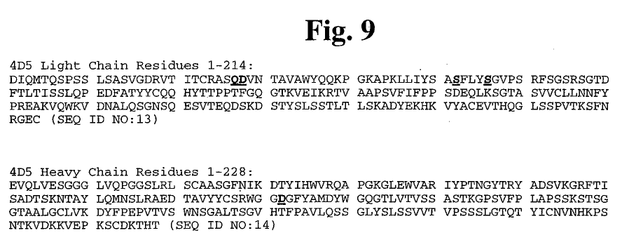

As the initial steps, a parent anti-TF antibody D3H44 (FIG. 8; SEQ ID NOS 11 and 12 for light and heavy chain variable domains, respectively) and a parent anti-HER2 antibody 4D5 (FIG. 9; SEQ ID NOS 13 and 14 for light and heavy chain variable domains, respectively) were used to identify potential ON-RAMPS, using similar criteria and calculations as described in Example 1. Table 4 and Table 5 list the first set of residues as potential ON-RAMPS of anti-TF D3H44 and anti-HER24D5, respectively, as well as single mutations to each of these residues along with the calculated on-rate relative to wild type. The calculated on-rate was calculated according to the method of Schreiber et al. (2000) Nat. Struct. Biol. 7:537-41. Further refinements, mutations and identification of additional ON-RAMPS are carried on using the similar methods and calculations.

TABLE 4
Potential ON-RAMPS of D3H44 and Single Mutations

| Mutation | Calculated on-rate (realative to WT) |
|---|---|
| VL-K30M | 1.2 |
| VL-K30E | 1.5 |
| VL-Y49E | 1.5 |
| VL-Y50E | 2.0 |
| VL-S53D | 1.5 |
| VH-K30D | 1.7 |
| VH-K30E | 1.4 |
| VH-Q54E | 0.9 |
| VH-N56K | 0.5 |
| VH-K62E | 1.7 |
| VH-K62D | 1.7 |
| VH-Q64E | 1.7 |
| VH-A97D | 4.8 |

Residues are numbered according the Kabat system (Kabat et al. Sequences of Proteins of Immunological Interest, 5th Edition., National Institute of Health, Bethesda, Md. (1991)).

TABLE 5
Potential ON-RAMPS of D3H44 and Single Mutations

| Mutant | Calculated on-rate (relative to WT) |
|---|---|
| VL-Q27K | 1.5-1.6 |
| VL-D28K | 8.0-20.0 |
| VL-S52K | 1.8-2.3 |
| VL-S56K | 1.4-2.0 |
| VH-D98K | 4.1-6.6 |

Residues are numbered according the Kabat system (Kabat et al. Sequences of Proteins of Immunological Interest, 5th Edition., National Institute of Health, Bethesda, Md. (1991)).

Following the identification of ON-RAMPS and design of single or multiple mutations accordingly, the association, dissociation rates and the overall binding affinities of the resulting variants can be observed and calculated according to the methods described in Example 1.

Particularly for anti-TF variants, however, because the association of TF and anti-TF is too rapid to be observed in a stirred cuvette, thus the fluorescence emission intensity (λ_{excitation}=280 nm, 2 nm bandpass; λₑₘᵢₛₛᵢₒₙ>320 nm,) was measured using a stopped-flow spectrophotometer (Aviv). 50 μL of a 100 mM solution of anti-TF in 10 mM HEPES, pH 7.0, 25° C., was rapidly mixed with 50 μL of a solution containing either 0 nM, 100 nM, 200 nM, 300 nM, 400 nM, 500 nM, 600 nM, 700 nM, 800 nM, or 900 nM TF and the change in fluorescence was observed over a period of 2 sec. The rate of change in fluoroescence intensity was fit to a single exponential curve. The association rate was determined by plotting the observed rate as a function of TF concentration. The slope of that line is the association rate (in M⁻¹ sec⁻¹).

What is claimed is:

1. A method of making an antibody variant of a parent antibody specific to an antigen, comprising the following steps:
a) identifying a target amino acid residue within the variable domain of the parent antibody, said target residue being 1) an exposed residue in solution; 2) in or adjacent to a hypervariable region; and 3) within about 20 Å of the antigen when the parent antibody is bound thereto; and
b) substituting the target residue of step a) with a different replacement amino acid residue such that the charge complementarity between the antibody and antigen is increased.

2. The method of claim 1 wherein the target residue does not directly contact antigen when bound thereto.

3. The method of claim 1 wherein the target residue has at least about one third of its side chain surface area exposed to solvent.

4. The method of claim 1 wherein the target residue is within at least about 16 Å of the antigen when bound thereto.

5. The method of claim 1 wherein the parent antibody is a humanized, human or chimeric antibody.

6. The method of claim 1 wherein the parent antibody is an antibody fragment.

7. The method of claim 6 wherein the antibody fragment is a Fab fragment.

8. The method of claim 1 wherein the antibody variant has a stronger binding affinity for the antigen than the parent antibody.

9. The method of claim 8 wherein the binding affinity of the antibody variant is at least about two fold stronger than the binding affinity of the parent antibody.

10. The method of claim 1 wherein the antibody variant has a faster association rate with the antigen than the parent antibody.

11. The method of claim 10 wherein the association rate of the antibody variant is at least about five fold faster than the association rate of the parent antibody.

12. The method of claim 10 wherein the association rate of the antibody variant is at least about ten fold faster than the association rate of the parent antibody.

13. The method of claim 1 wherein the antibody variant has from about one to about twenty substitutions in the hypervariable regions thereof compared to the parent antibody.

14. The method of claim 13 wherein each of the substitutions increases charge complementarity between the antibody and antigen.

15. The method of claim 1 wherein the antigen is vascular endothelial growth factor (VEGF).

16. The method of claim 15 wherein the parent antibody comprises the heavy and light chain variable domains of a humanized anti-VEGF antibody selected from the group consisting of Y0101, Y0317, F(ab)-12, Y0192, Y0238-3, Y0239-19, Y0313-2, and VNERK.

17. The method of claim 1 wherein the substitution is in a hypervariable region selected from the group consisting of CDR L1, CDR L2, loop H1 and CDR H3.

18. The method of claim 16 wherein the substitution is at one or more of amino acid positions 26L, 27L, 28L, 30L, 31L, 32L, 50L, 52L, 53L, 54L, 56L, 93L or 94L of a light chain variable domain of the parent antibody, utilizing the residue numbering system according to Kabat.

19. The method of claim 18 wherein the substitution is at two or more of amino acid positions 26L, 27L, 28L or 30L of a light chain variable domain of the parent antibody, utilizing the residue numbering system according to Kabat.

20. The method of claim 19 wherein the substitution is at three or four of amino acid positions 26L, 27L, 28L or 30L of a light chain variable domain of the parent antibody, utilizing the residue numbering system according to Kabat.

21. The method of claim 16 wherein the substitution is at one or more of amino acid positions 25H, 28H, 30H, 54H, 56H, 61H, 62H, 99H or 100aH of a heavy chain variable domain of the parent antibody, utilizing the residue numbering system according to Kabat.

22. The method of claim 1, wherein the antigen is tissue factor (TF).

23. The method of claim 22, wherein the parent antibody comprises the heavy and light chain variable domains of a humanized anti-TF antibody.

24. The method of claim 23, wherein the humanized anti-TF antibody is D3H44.

25. The method of claim 23, wherein the substitution is at least at one or more of amino acid positions 30L, 49L, 50L, 53L of a light chain variable domain of the parent antibody, utilizing the residue numbering system according to Kabat.

26. The method of claim 25, wherein the light chain variable domain of the parent antibody is of SEQ ID NO:11.

27. The method of claim 23 wherein the substitution is at least at one or more of amino acid positions 30H, 54H, 56H, 62H, 64H or H of a heavy chain variable domain of the parent antibody, utilizing the residue numbering system according to Kabat.

28. The method of claim 27, wherein the heavy chain variable domain of the parent antibody is of SEQ ID NO:12.

29. The method of claim 1, wherein the antigen is HER2.

30. The method of claim 29, wherein the parent antibody comprises the heavy and light chain variable domains of a humanized anti-HER2 antibody.

31. The method of claim 30, wherein the humanized anti-HER2 antibody is the rhuMAb 4D5.

32. The method of claim 30, wherein the substitution is at least at one or more of amino acid positions 27L, 28L, 52L or 56L of a light chain variable domain of the parent antibody, utilizing the residue numbering system according to Kabat.

33. The method of claim 32, wherein the light chain variable domain of the parent antibody is of SEQ ID NO:13.

34. The method of claim 30 wherein the substitution is at least at amino acid position 98H of a heavy chain variable domain of the parent antibody, utilizing the residue numbering system according to Kabat.

35. The method of claim 34, wherein the heavy chain variable domain of the parent antibody is of SEQ ID NO:14.

36. The method of claim 1 comprising producing the antibody variant in a host cell comprising nucleic acid encoding the antibody variant.

37. The method of claim 36 comprising conjugating the antibody variant produced by the host cell with a heterologous molecule.

38. An antibody variant made according to the method of claim 36.

39. An antibody variant of a parent antibody which comprises an amino acid alteration in or adjacent to a hypervariable region of the parent antibody which increases charge complementarity between the antibody variant and an antigen to which it binds.

40. The antibody variant of claim 39 wherein the alteration is an amino acid substitution in a hypervariable region of the parent antibody.

41. The antibody variant of claim 39 wherein the alteration is an amino acid insertion in or adjacent to a hypervariable region of the parent antibody, wherein the inserted amino acid does not bind antigen.

42. The antibody variant of claim 39 wherein the antigen is vascular endothelial growth factor (VEGF).

43. The antibody variant of claim 42 comprising a light chain variable domain comprising a CDR L1 sequence selected from SATKKIKNYLN (SEQ ID NO:6) or SATKKITNYLN (SEQ ID NO:7).

44. The antibody variant of claim 43 comprising a light chain variable domain comprising the amino acid sequence of SEQ ID NO:3 or SEQ ID NO:4.

45. The antibody variant of claim 42 comprising a heavy chain variable domain comprising the amino acid sequence of SEQ ID NO:5, SEQ ID NO:8, SEQ ID NO:9 or SEQ ID NO:10.

46. The antibody variant of claim 39 wherein the antigen is tissue factor (TF).

47. The antibody variant of claim 46 wherein the parent antibody is D3H44.

48. The antibody variant of claim 39 wherein the antigen is HER2.

49. The antibody variant of claim 48 wherein the parent antibody is 4D5.

50. A composition comprising the antibody variant of claim 39 and a pharmaceutically acceptable carrier.

51. Isolated nucleic acid encoding the antibody variant of claim 39.

52. A vector comprising the nucleic acid of claim 51.

53. A host cell transformed with the nucleic acid of claim 51.

54. A process of producing an antibody variant comprising culturing the host cell of claim 53 so that the nucleic acid is expressed.

55. The process of claim 54 further comprising recovering the antibody variant from the host cell culture.

56. The process of claim 55 wherein the antibody variant is recovered from the host cell culture medium.

57. A method for determining antigen association rate of an antibody comprising:
(1) combining antibody and antigen in solution, and then;
(2) determining formation of antibody-antigen complex over time.

58. The method of claim 57 wherein step (2) comprises measuring fluorescence emission intensity of the antibody-antigen complex.

59. The method of claim 57 wherein the antibody or antigen comprises a tryptophan residue at the antigen-antibody binding interface, and step (2) measures fluorescence emission intensity of the tryptophan residue which changes when the tryptophan residue is buried.

60. The method of claim 57 wherein the antigen is vascular endothelial growth factor.

61. The method of claim 57 wherein the antibody has an association constant for antigen slower than 10⁵ M⁻¹ sec⁻¹.
